(19) United States
(12) Patent Application Publication
(10) Pub. No.: US 2008/0208190 A1
(43) Pub. Date: Aug. 28, 2008

(54) HAIR REMOVAL APPARATUS

(75) Inventors: Tetsuro HASHIGUCHI, Hikone (JP); Kenichi MURAKI, Hikone (JP)

Correspondence Address: GREENBLUM & BERNSTEIN, P.L.C., 1950 ROLAND CLARKE PLACE, RESTON, VA, 20191, US

(73) Assignee: MATSUSHITA ELECTRIC WORKS, LTD., Osaka (JP)

(21) Appl. No.: 12/029,617

(22) Filed: Feb. 12, 2008

(30) Foreign Application Priority Data
Feb. 23, 2007 (JP) 2007-043359

(51) Int. Cl. A61B 18/00; A61B 17/50

(52) U.S. Cl. 606/43; 606/133; 606/210; 606/32

(57) ABSTRACT

A hair removal apparatus includes a plurality of plucking rollers configured to rotate on respective roller shafts which extend generally parallel to a skin surface during epilation, each plucking roller having tweezing mechanisms for grasping hairs, and a driving device for turning each plucking roller on a rotary axis which is kept generally perpendicular to the skin surface. The driving device turns each of the plucking rollers in such a way that the rotary axis thereof assess approximately a central point of the relevant plucking roller.

### BACKGROUND OF THE INVENTION

1. Field of the Invention

The present invention relates to a hair removal apparatus for removing unwanted body hair by pulling the hair out of the skin.

2. Description of the Related Art

Japanese Unexamined Patent Publication No. 1998-151020 describes a conventionally known example of a hair removal apparatus provided with an extractor for pulling hairs out of human skin by sliding the apparatus along the skin surface so that the extractor grips and removes the hairs. FIG. 12 is a perspective view of the hair removal apparatus proposed in the aforementioned Publication. This hair removal apparatus includes a plurality of tweezing discs (i.e., extractor) 3 mounted on unillustrated multiple rotating plates which are fixed to a rotary shaft 62 to extend radially outward therefrom. The tweezing discs 3 on each rotating plate are configured to turn on an axis thereof and rotate around the rotary shaft 62 as a result of orbital motion of the rotating plates around the rotary shaft 62 while moving along the skin surface and uprooting the hairs.

In an epilation method in which the extractor grips and pulls out hairs from the skin, it is known that the extractor can pluck the hairs more easily when slid against a direction of hair growth to make the hairs to stand erect than in the hair growth direction causing the hairs to lie flat. Thus, the hair removal apparatus achieves higher epilation efficiency when moved in strokes against the hair growth direction.

When using the conventional hair removal apparatus like the one mentioned above, a user becomes accustomed to constantly move the apparatus so that the extractor would slide against the hair growth direction on the skin surface. In practice, however, although the user can slide the extractor against the hair growth direction in most areas of the skin in this way, the extractor will move along the hair growth direction or in other directions in some local skin areas where the direction of hair growth is nonuniform. Thus, the conventional hair removal apparatus has a chronic problem that areas of high and low epilation efficiencies occur on the user's epilated body part.

### SUMMARY OF THE INVENTION

The present invention is intended to provide a solution to the aforementioned problem of the prior art. Accordingly, it is an object of the invention to provide a hair removal apparatus which can offer an improved epilation efficiency uniformly over an area of contact between an extractor and a skin surface to be treated.

According to an aspect of the invention, a hair removal apparatus comprises an extractor which rotates on a first rotary axis extending in a direction generally parallel to a skin surface during epilation, the extractor having a tweezing mechanism for grasping hairs, and a driver for turning the extractor on a second rotary axis which is kept generally perpendicular to the skin surface. The driver turns the extractor in such a way that the second rotary axis passes approximately a central point of the extractor, and the hair removal apparatus pulls the grasped hairs out of the skin surface when moved with the extractor held in contact with the skin surface.

These and other objects, features and advantages of the invention will become more apparent upon a reading of the following detailed description in conjunction with the accompanying drawings.

### BRIEF DESCRIPTION OF THE DRAWINGS

FIG. 1 is a perspective view of a hair removal apparatus according to a preferred embodiment of the invention;

FIG. 2 is a side view of the hair removal apparatus shown in FIG. 1;

FIG. 3 is an exploded perspective view of the hair removal apparatus shown in FIG. 1;

FIG. 4 is a cross-sectional view of the hair removal apparatus taken along lines IV-IV shown in FIG. 1;

FIG. 5 is a cross-sectional view of the hair removal apparatus taken along lines V-V shown in FIG. 4;

FIG. 6 is a perspective view of a plucking roller of one of epilation units shown in FIG. 4;

FIG. 7 is a perspective view showing how motive power is transmitted by a first motive power transmission mechanism of the hair removal apparatus;

FIG. 8 is a perspective view showing how the motive power is transmitted by a second motive power transmission mechanism of the hair removal apparatus;

FIG. 9 is a side view showing the epilation units under normal conditions;

FIG. 10 is a side view showing the epilation units under floated conditions;

FIG. 11 is a cross-sectional view corresponding to FIG. 4 showing one of the epilation units under the floated conditions; and

FIG. 12 is a perspective view of a conventional hair removal apparatus.

### DETAILED DESCRIPTION OF THE PREFERRED EMBODIMENT OF THE INVENTION

A preferred embodiment of the invention is now described in detail with reference to the accompanying drawings.

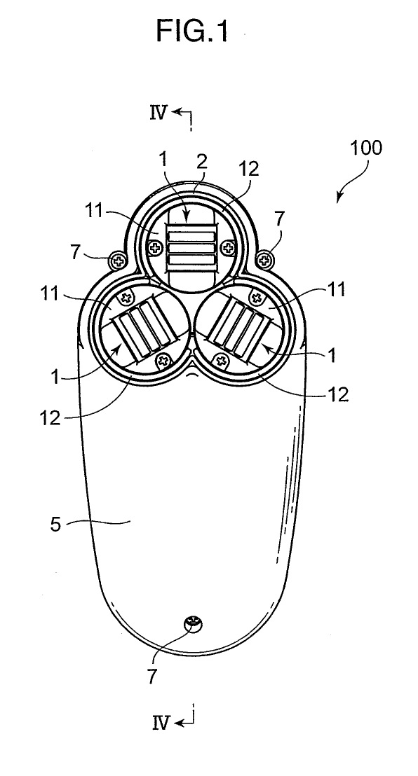

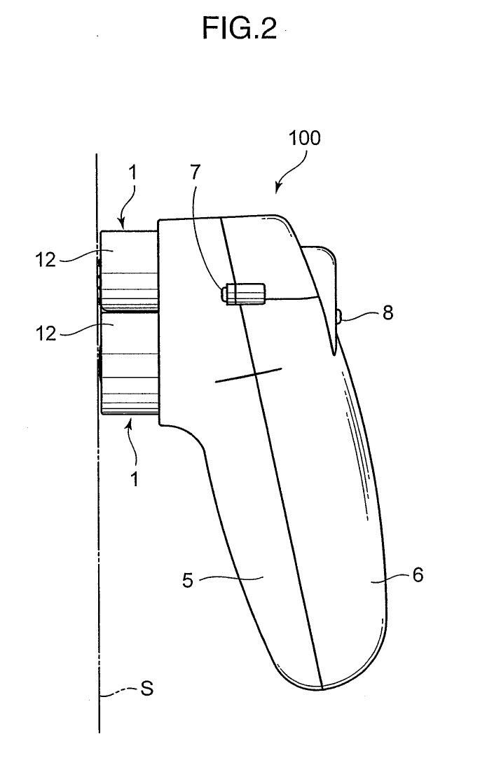

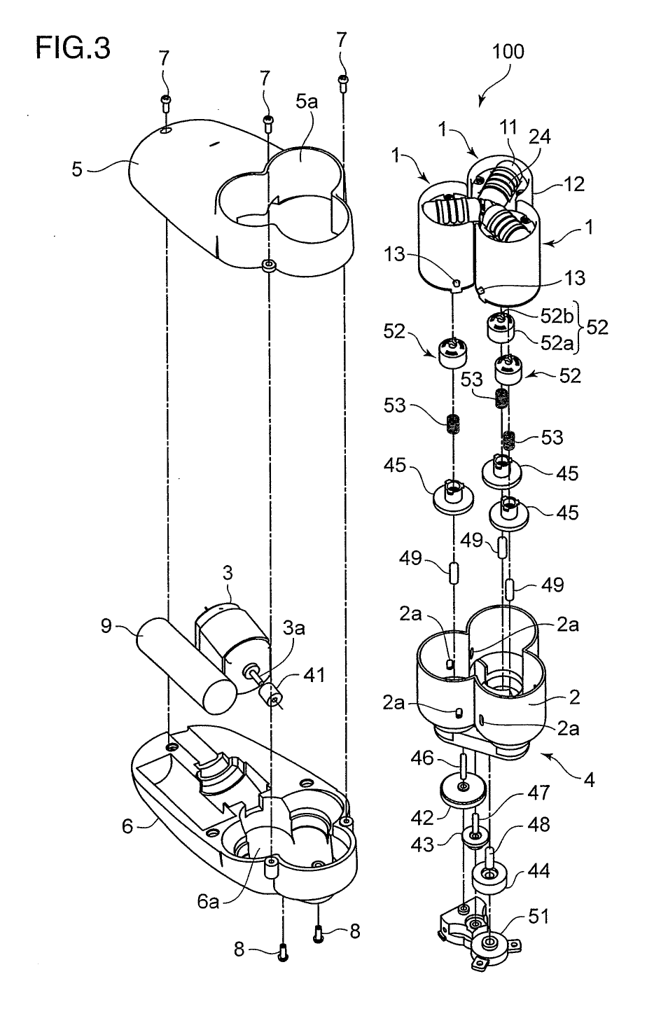

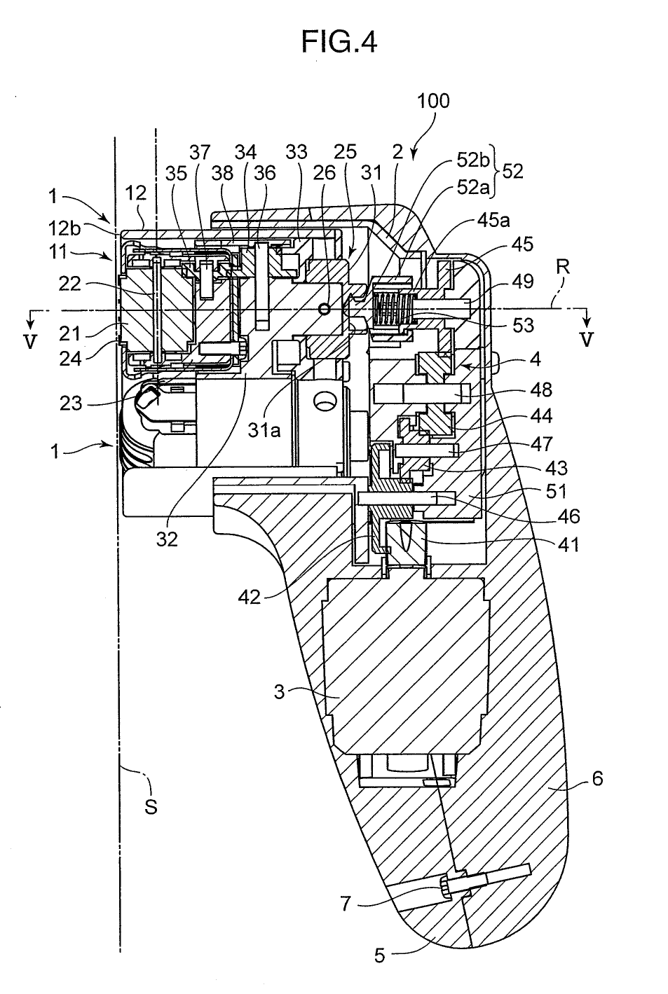

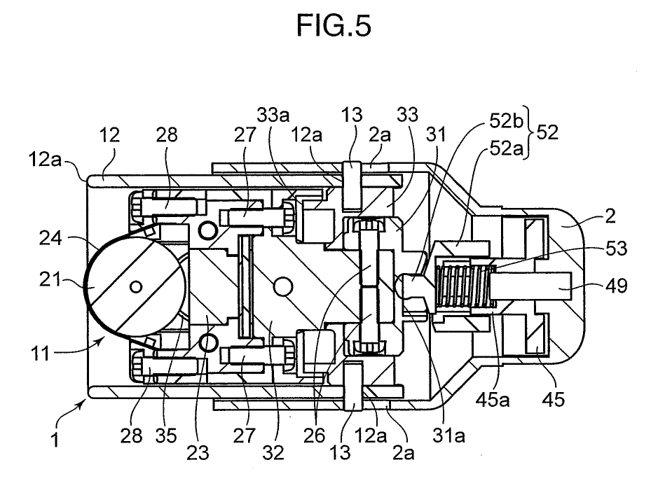

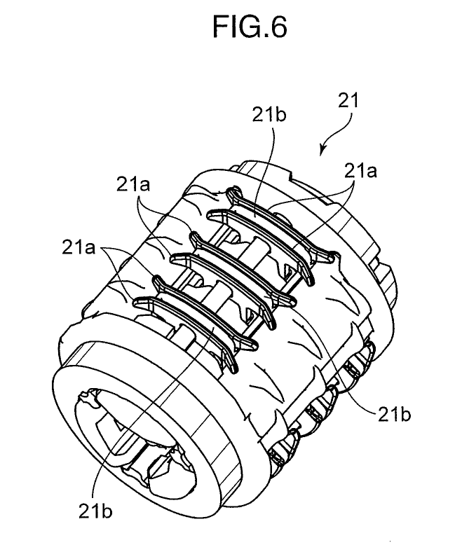

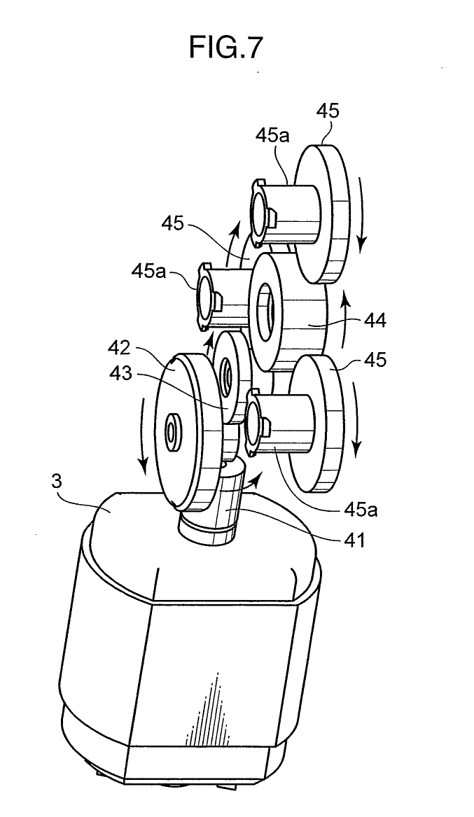

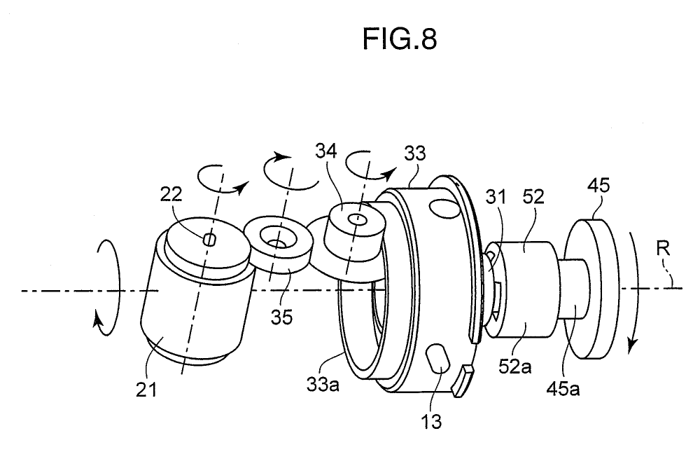

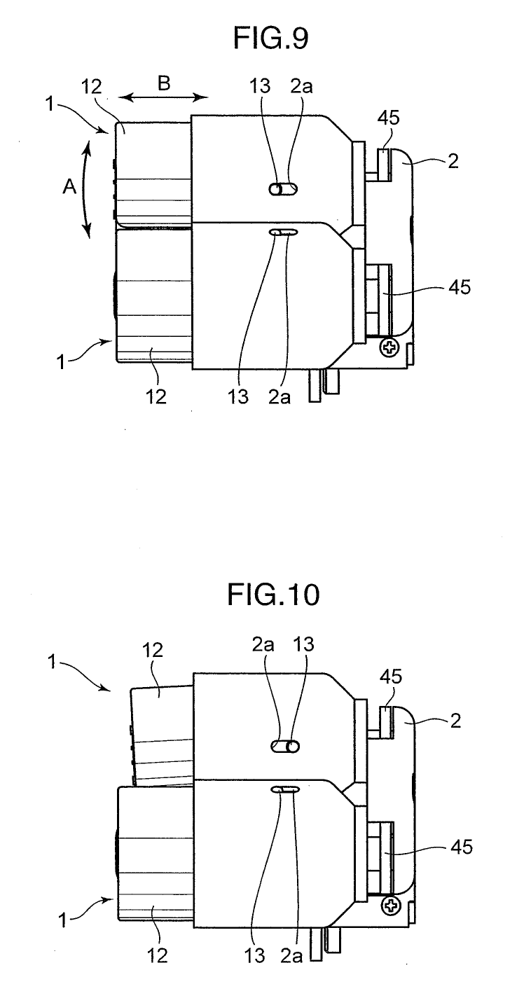

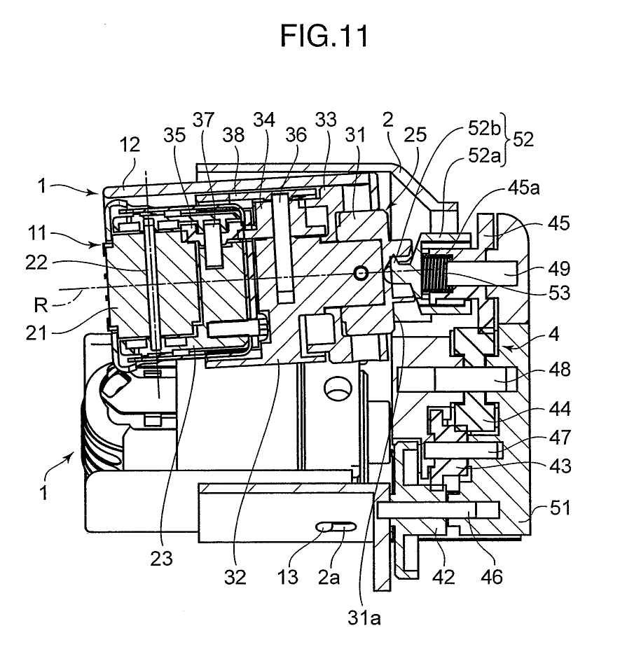

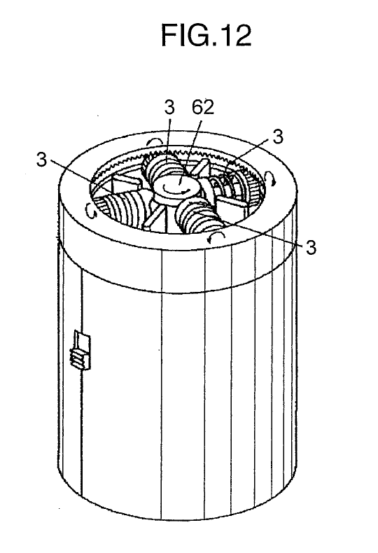

FIGS. 1 and 2 are a perspective view and a side view of a hair removal apparatus 100 according to a preferred embodiment of the invention, respectively, and FIG. 3 is an exploded perspective view of the hair removal apparatus 100 shown in FIG. 1. FIGS. 4 and 5 are cross-sectional views of the hair removal apparatus 100, and FIG. 6 is a perspective view of a plucking roller 21 of one of epilation units 1. FIGS. 7 and 8 are perspective views showing how motive power is generated and transmitted for driving the hair removal apparatus 100 and FIGS. 9 and 10 are side views showing the epilation units 1 under normal conditions and floated conditions, respectively. FIG. 11 is a cross-sectional view corresponding to FIG. 4 showing the epilation unit 1 under the floated conditions.

The hair removal apparatus 100 is configured to pull out unwanted body hairs, in particular those from arms, legs and underarms. Referring to FIGS. 1 to 3, the hair removal apparatus 100 includes three epilation units 1, a supportive housing 2 for supporting the three epilation units 1, a motor 3 serving as a power source, a first motive power transmission mechanism 4 for transmitting a rotational driving force generated by the motor 3 to the epilation units 1, and a pair of outer covers 5, 6 incorporating all internal elements of the hair removal apparatus 100 together forming a structure grippable by a single hand. The two outer covers 5, 6 are fixed to each other by screws 7. There is formed an opening 5a in the outer cover 5 so that the three epilation units 1 are exposed outward toward a skin surface S as shown in FIG. 2. The motor 3 is connected to a battery 9 serving as a power supply.

The three epilation units 1 are closely located in such a way that central points of the epilation units 1 are positioned at vertices of a generally equilateral triangle in plan view as depicted in FIG. 1. Also, the three epilation units 1 are disposed to project toward the skin surface S generally by the same amount as depicted in FIG. 2. As shown in FIGS. 3 to 5, each of the epilation units 1 includes a rotor 11, a sleeve 12 and a pair of pins 13 serving as a pivotal axis.

The rotor 11 is configured to rotate on an axis R shown in FIG. 4 due to the rotational driving force transmitted through the first motive power transmission mechanism 4 with the axis R kept generally perpendicular to the skin surface S. In this embodiment, the axis R of the rotor 11 passes approximately a central point of the plucking roller 21. The rotor 11 of each epilation unit 1 includes the aforementioned plucking roller 21, a roller shaft 22, a roller base 23, a protective plate 24 and a second motive power transmission mechanism 25.

Each of the plucking rollers 21 is supported rotatably on its own axis by the roller shaft 22. The plucking roller 21 of each rotor 11 carries a plurality of tweezing mechanisms 21a fitted on a curved outer surface, each tweezing mechanism 21a including a pair of platelike members arranged to extend side by side along a rotating direction of the plucking roller 21 with a narrow gap 21b formed between the platelike members to guide body hairs thereinto as shown in FIG. 6. These tweezing mechanisms 21a are configured to close the gaps 21b when brought into contact with the skin surface S, whereby the tweezing mechanisms 21a grasp the body hairs introduced into the gaps 21b.

The roller shaft 22 of each rotor 11 extends in a direction generally parallel to the skin surface S and each plucking roller 21 supported by the roller shaft 22 rotates on its own axis during epilation. When a user moves the hair removal apparatus 100 along the skin surface S, the rotating plucking rollers 21 grasp and pull out the body hairs.

As shown in FIG. 5, the roller base 23 supporting the roller shaft 22 of each rotor 11 is affixed to a later-described roller case 32 by screws 27.

The protective plates 24 of the rotors 11 lie on areas of the skin surface S with which the individual plucking rollers 21 go into contact. The protective plates 24 serve to protect the skin surface S by preventing the tweezing mechanisms 21a from contacting the skin surface S with excessive pressure. Each protective plate 24 is affixed to the roller base 23 by screws 28.

The second motive power transmission mechanism 25 of each rotor 11 is connected to the first motive power transmission mechanism 4 to transmit the rotational driving force supplied therefrom to the plucking roller 21 for rotating the same. As shown in FIG. 4, the second motive power transmission mechanism 25 includes a power input member 31, the aforementioned roller case 32, a crown gear 33 and epilation unit-side gears 34, 35.

The power input member 31 of each second motive power transmission mechanism 25 is a mechanical element to which the rotational driving force supplied from the first motive power transmission mechanism 4 is first transmitted to the epilation unit 1. Each power input member 31 has a mating part 31a recessed in a U-shaped cross section which can flexibly engage a projecting part 52b of each of later-described cylindrical members 52 of the first motive power transmission mechanism 4. The power input member 31 thus configured turns on the aforementioned axis R due to the rotational driving force input from the first motive power transmission mechanism 4.

As the roller case 32 of each second motive power transmission mechanism 25 is joined to the power input member 31 by screws 26, the roller case 32 rotates together with the power input member 31. Also, as the roller base 23 is affixed to the roller case 32 by the screws 27 as mentioned above, the power input member 31, the roller case 32, the roller base 23 and the plucking roller 21 rotate as a single structure on the axis R.

The crown gear 33 of each epilation unit 1 is fitted with the pair of pins 13. These pins 13 are fitted into pin holes 12a formed in the sleeve 12 and into later-described slots 2a formed in the supportive housing 2 so that the crown gear 33 is a nonrotating element. Further, the crown gear 33 has a face gear portion 33a which meshes with the epilation unit-side gear 34. The epilation unit-side gear 34 is pivotably supported by a gear shaft 36 fitted between a side portion of the roller case 32 and a gear cover 38. In the epilation unit 1 thus configured, the epilation unit-side gear 34 turns along the face gear portion 33a of the crown gear 33 when the roller case 32 rotates together with the power input member 31.

The other epilation unit-side gear 35 mates with the aforementioned epilation unit-side gear 34 and is pivotably supported by a gear shaft 37 fitted on a side portion of the roller base 23. This epilation unit-side gear 35 also mates with an unillustrated gear portion formed on an end portion of the plucking roller 21 and thus serves to transmit the rotational driving force to the plucking roller 21 so that the plucking roller 21 can rotate on the roller shaft 22.

The sleeve 12 of each epilation unit 1 surrounds the plucking roller 21 and projects toward the skin surface S. In this embodiment, the sleeve 12 is configured such that an outer end 12b thereof placed against the skin surface S becomes flush with an extreme outer part of the plucking roller 21 that is brought into contact with the skin surface S as shown in FIG. 4. Thus, the individual sleeves 12 go into contact with the skin surface S together with the plucking rollers 21. This arrangement of the embodiment may be modified such that the outer ends 12b of the sleeves 12 slightly project further outward toward the skin surface S than the extreme outer parts of the plucking rollers 21.

The aforementioned supportive housing 2 has a triple-cup structure for accommodating the three epilation units 1. The aforementioned slots 2a in which the pairs of pins 13 of the individual epilation units 1 are fitted are formed in the supportive housing 2 at appropriate points thereof so that each epilation unit 1 can swing in a direction marked by “A” in FIG. 9 with respect to the supportive housing 2. Also, the slots 2a in the supportive housing 2 are formed to create some play for the pins 13 of each epilation unit 1 to move along a direction marked by “B” in FIG. 9 so that each epilation unit 1 can float (i.e., protrude and retract) along the B direction with respect to the supportive housing 2. This arrangement of the embodiment enables the individual epilation units 1 to float up and down as shown in FIG. 10 while swinging independently of one another.

Referring to FIG. 7, the first motive power transmission mechanism 4 includes driving and transmission gears 41-45 and gear shafts 46-49 rotatably supporting the gears 42-45, respectively, which are arranged in this order from a driving side to a driven side. The first motive power transmission mechanism 4 further includes a gear cover 51, the aforementioned cylindrical members 52 and compression coil springs 53.

The driving gear 41 is fitted on a driving shaft 3a of the motor 3. The gears 42-44 are rotatably supported by the respective gear shafts 46-48 of which opposite ends are supported by the supportive housing 2 and the gear cover 51. With the first motive power transmission mechanism 4 thus configured, the motive power of the motor 3 output from the driving shaft 3a is transmitted to the individual epilation units 1 through three driving gears 45. The aforementioned gear cover 51 is fixed to an inside of a recessed part 6a of the outer cover 6 by screws 8.

The three driving gears 45 fitted on the gear shafts 49 which are mounted in the supportive housing 2 are configured to engage the respective cylindrical members 52. Specifically, as gear teeth formed on a curved outer surface of a projecting sleevelike part 45a of each driving gear 45 mesh with gear teeth formed on a curved inner surface of a sleevelike part 52a of the corresponding cylindrical member 52 as shown in FIG. 5, the motive power of the motor 3 is transmitted to each cylindrical member 52. The compression coil spring 53 is fitted in the sleevelike part 52a of the cylindrical member 52 of each epilation unit 1 to produce a biasing force acting on the gear shaft 45 and the cylindrical member 52 in opposite directions, causing both to separate from each other. This configuration makes it possible to easily bring each epilation unit 1 which has retracted into the supportive housing 2 back to an original position.

Each of the cylindrical members 52 of the first motive power transmission mechanism 4 has the aforementioned projecting part 52b which meshes with the recessed mating part 31a of the corresponding power input member 31. The projecting part 52b of each cylindrical member 52 has a generally triangular shape in plan view. The mating part 31a of each power input member 31 also has a generally triangular shape in plan view although not illustrated. This arrangement ensures that the epilation units 1 will not easily be disengaged from the supportive housing 2 regardless of floating and/or swinging action of the epilation units 1 relative to the supportive housing 2 (refer to FIG. 11).

In the hair removal apparatus 100 thus configured, the motive power (rotational driving force) produced by the motor 3 is transmitted to the three driving gears 45 through the gears 41-45 of the first motive power transmission mechanism 4 (refer to FIG. 7) and then further downstream to the power input members 31 of the epilation units 1 via the respective cylindrical members 52 (refer to FIG. 8), thereby causing the rotors 11 of the epilation units 1 to rotate on the respective axes R.

Since the cylindrical members 52 of the first motive power transmission mechanism 4 are connected to the power input members 31 with the projecting parts 52b flexibly meshed with the respective mating parts 31a as mentioned above, the cylindrical members 52 and the power input members 31 are securely held in an interconnected state even when the epilation units 1 are making swing motion in the supportive housing 2. Additionally, since the cylindrical members 52 incorporate the compression coil springs 53 for biasing the cylindrical members 52 toward the respective power input members 31, the cylindrical members 52 can float up and down relative to the respective driving gears 45. This structure of the embodiment makes it possible to reliably transmit the motive power (rotational driving force) to the epilation units 1 even when the epilation units 1 swing and float to a great extent relative to the supportive housing 2.

Since the epilation unit-side gears 34 built in the rotors 11 mesh with the face gear portions 33a of the respective crown gears 33, the epilation unit-side gears 34 rotate on their own axes when the rotors 11 turn on the respective axes R. Also, as the rotational driving force of the first motive power transmission mechanism 4 is transmitted to the individual plucking rollers 21 via the epilation unit-side gears 34, 35, the plucking rollers 21 rotate on the respective roller shafts 22.

As thus far described, the three epilation units 1 having the capability to swing and float independently of one another incorporate the rotors 11 of which plucking rollers 21 turn on the respective axes R while rotating on the respective roller shafts 22. According to this structure, the hair removal apparatus 100 of the embodiment can pluck the body hairs from the skin while varying the direction of movement of the tweezing mechanisms 21a of each plucking roller 21 relative to the skin surface S as the plucking rollers 21 turn on the respective axes R.

In the above-described embodiment of the invention, the plucking rollers 21 rotating on their own axes, or on the roller shafts 22 disposed generally parallel to the skin surface S during epilation, are caused to turn on the axes R passing approximately the central points of the respective plucking rollers 21 generally at right angles to the skin surface S. This arrangement makes it possible to vary the direction of movement of the tweezing mechanisms 21a relative to the skin surface S as the plucking rollers 21 rotate on the respective axes R. When any of the plucking rollers 21 has rotated by approximately 180 degrees, for example, the direction of movement of the tweezing mechanisms 21a of that plucking roller 21 relative to the skin surface S is nearly reversed. It is conventionally known that the body hairs can be easily guided and gripped in between the facing platelike members of the tweezing mechanisms 21a when the tweezing mechanisms 21a move against a direction of hair growth as compared to a case where the tweezing mechanisms 21a move along the direction of hair growth. According to the aforementioned structure of the present embodiment, the tweezing mechanisms 21a of each plucking roller 21 move against the hair growth direction at some timing during every rotation of each plucking roller 21 on the axis R. Therefore, the hair removal apparatus 100 of the embodiment can create a desirable state for epilating unwanted body hairs in all contact areas between the plucking rollers 21 and the skin surface S.

In this embodiment, a driving device for turning each epilation unit 1 on the axis R serves also as means for rotating the plucking roller 21 on the roller shaft 22 so that it is possible to prevent complication of a mechanism for driving the plucking roller 21.

In this embodiment, the three epilation units 1 are closely positioned in such a way that the central points of the epilation units 1 are located at the vertices of a generally equilateral triangle in plan view as mentioned earlier, so that each plucking roller 21 is located in a space created between the two other adjacent plucking rollers 21 as if overlapping the latter two plucking rollers 21. This arrangement is advantageous in that the hair removal apparatus 100 is less likely to leave untreated areas on the skin surface S when moved therealong. Hence, the hair removal apparatus 100 can be used with substantially no lowering of epilation efficiency even when a total epilation area increases.

In this embodiment, the sleeve 12 of each epilation unit 1 is structured such that the outer end 12b of the sleeve 12 placed against the skin surface S becomes flush with the extreme outer part of the plucking roller 21 that is brought into contact with the skin surface S as mentioned earlier, so that the individual sleeves 12 go into contact with the skin surface S together with the plucking rollers 21. This arrangement is advantageous for preventing such inconvenience that the hair removal apparatus 100 is led to unintended directions on the skin surface S due to strong contact of only the rotating plucking rollers 21 with the skin, thereby enabling smooth epilation. Additionally, this arrangement of the embodiment serves to alleviate an unpleasant feeling that the user may have when the hair is pulled out of the skin because the sleeves 12 hold the skin tight during epilation.

Furthermore, the hair removal apparatus 100 of the embodiment employs a modular design in which the plucking roller 21 and the second motive power transmission mechanism 25 are compactly assembled and built in each of the epilation units 1 as thus far described, so that the hair removal apparatus 100 can be redesigned to accommodate a desired number of epilation units 1 other than three without any extensive design change in the aforementioned structure of the driving device.

While the invention has thus been described with reference to the hair removal apparatus 100 of the preferred embodiment provided with the three epilation units 1, the invention is not limited to this embodiment but is similarly applicable to a hair removal apparatus provided with one or two epilation units or more than three epilation units.

Also, while the hair removal apparatus 100 of the foregoing embodiment is configured such that each of the plucking rollers 21 rotates on its own axis provided by the roller shaft 22 due to the rotational driving force transmitted by the first motive power transmission mechanism 4, the invention is not limited to this configuration but may be modified such that rotation of each of the plucking rollers 21 on its own axis is produced by a different type of driving mechanism.

The invention has thus far been described in detail, by way of example, with reference to the preferred embodiment thereof.

In summary, a hair removal apparatus comprises an extractor which rotates on a first rotary axis extending in a direction generally parallel to a skin surface during epilation, the extractor having a tweezing mechanism for grasping hairs, and a driver for turning the extractor on a second rotary axis which is kept generally perpendicular to the skin surface. The driver turns the extractor in such a way that the second rotary axis passes approximately a central point of the extractor, and the hair removal apparatus pulls the grasped hairs out of the skin surface when moved with the extractor held in contact with the skin surface.

In the hair removal apparatus thus configured, the extractor rotating on its own axis (first rotary axis) generally parallel to the skin surface is caused to turn on the second rotary axis passing approximately the central point of the extractor generally at right angles to the skin surface. This arrangement makes it possible to vary the direction of movement of the tweezing mechanism relative to the skin surface as the extractor rotates on the second rotary axis. When the extractor has rotated by approximately 180 degrees, for example, the direction of movement of the tweezing mechanism relative to the skin surface is nearly reversed. It is conventionally known that the body hairs can be easily guided and gripped in between facing members of the tweezing mechanism when the tweezing mechanism moves against a direction of hair growth as compared to a case where the tweezing mechanism moves along the direction of hair growth. According to the aforementioned structure, the tweezing mechanism moves against the hair growth direction at some timing during every rotation of the extractor on the second rotary axis. Therefore, the hair removal apparatus can create a desirable state for epilating unwanted body hairs in all contact areas between the extractor and the skin surface.

The hair removal apparatus may be configured such that the extractor is driven to rotate on the first rotary axis by a driving force supplied from the driver.

In the hair removal apparatus thus configured, the driver for turning the extractor on the second rotary axis serves also as means for rotating the extractor on the first rotary axis so that it is possible to prevent complication of a mechanism for driving the extractor.

The he hair removal apparatus further comprises a sleeve which surrounds the extractor and projects outward toward the skin surface at least to a point where an outer end of the sleeve becomes flush with an extreme outer part of the extractor.

In the hair removal apparatus thus configured, the sleeve is structured such that the outer end thereof placed against the skin surface becomes flush with the extreme outer part of the extractor that is brought into contact with the skin surface, so that the sleeve goes into contact with the skin surface together with the extractor. This arrangement is advantageous for preventing such inconvenience that the hair removal apparatus is led to unintended directions on the skin surface due to strong contact of the rotating extractor with the skin, thereby enabling smooth epilation. Additionally, this arrangement serves to alleviate an unpleasant feeling that the user may have when the hair is pulled out of the skin because the sleeve holds the skin tight during epilation.

The extractor may include three extracting parts which are closely located in such a way that central points of the individual extracting parts are positioned at vertices of a generally equilateral triangle in plan view.

In the hair removal apparatus thus configured, the three extracting parts are closely positioned in such a way that the central points of the extracting parts are located at the vertices of a generally equilateral triangle in plan view, so that each extracting part is located in a space created between the two other adjacent extracting parts as if overlapping the latter two extracting parts. This arrangement is advantageous in that the hair removal apparatus is less likely to leave untreated areas on the skin surface when moved therealong. Hence, the hair removal apparatus can be used with substantially no lowering of epilation efficiency even when a total epilation area increases.

The driver may include a first motive power transmission mechanism connected to a power source, and second motive power transmission mechanisms connected to the first motive power transmission mechanism to transmit the driving force supplied therefrom to the individual extracting parts, wherein each of the extracting parts and the corresponding second motive power transmission mechanism are assembled to together constitute a modular epilation unit.

According to this feature, the hair removal apparatus can be redesigned to accommodate a desired number of epilation units other than three without any extensive design change in the aforementioned structure of the driver.

This application is based on Japanese Patent Application No. 2007-043359, filed in Japan Patent Office on Feb. 23, 2007, the contents of which are hereby incorporated by reference.

Although the present invention has been fully described by way of example with reference to the accompanied drawings, it is to be understood that various changes and modifications will be apparent to those skilled in the art. Therefore, unless otherwise such changes and modifications depart from the scope of the present invention hereinafter defined, they should be construed as being included therein.

What is claimed is:

1. A hair removal apparatus comprising:
an extractor which rotates on a first rotary axis extending in a direction generally parallel to a skin surface during epilation, the extractor having a tweezing mechanism for grasping hairs; and
a driver for turning the extractor on a second rotary axis which is kept generally perpendicular to the skin surface;
wherein the driver turns the extractor in such a way that the second rotary axis passes approximately a central point of the extractor, and the hair removal apparatus pulls the grasped hairs out of the skin surface when moved with the extractor held in contact with the skin surface.

2. The hair removal apparatus according to claim 1, wherein the extractor is driven to rotate on the first rotary axis by a driving force supplied from the driver.

3. The hair removal apparatus according to claim 2, further comprising a sleeve which surrounds the extractor and projects outward toward the skin surface at least to a point where an outer end of the sleeve becomes flush with an extreme outer part of the extractor.

4. The hair removal apparatus according to claim 3, wherein the extractor includes three extracting parts which are closely located in such a way that central points of the individual extracting parts are positioned at vertices of a generally equilateral triangle in plan view.

5. The hair removal apparatus according to claim 4, wherein the driver includes:
a first motive power transmission mechanism connected to a power source; and
a plurality of second motive power transmission mechanisms connected to the first motive power transmission mechanism to transmit the driving force supplied therefrom to the individual extracting parts;
wherein each of the extracting parts and the corresponding second motive power transmission mechanism are assembled to together constitute a modular epilation unit.

6. The hair removal apparatus according to claim 2, wherein the extractor includes three extracting parts which are closely located in such a way that central points of the individual extracting parts are positioned at vertices of a generally equilateral triangle in plan view.

7. The hair removal apparatus according to claim 1, further comprising a sleeve which surrounds the extractor and projects outward toward the skin surface at least to a point where an outer end of the sleeve becomes flush with an extreme outer part of the extractor.

8. The hair removal apparatus according to claim 1, wherein the extractor includes three extracting parts which are closely located in such a way that central points of the individual extracting parts are positioned at vertices of a generally equilateral triangle in plan view.
